(19) United States
(12) Patent Application Publication
(10) Pub. No.: US 2020/0270576 A1
(43) Pub. Date: Aug. 27, 2020

(54) Ex Vivo Maintenance and Expansion of Stem Cells

(71) Applicant: THE REGENTS OF THE UNIVERSITY OF CALIFORNIA, Oakland, CA (US)

(72) Inventor: Robert Signer, La Jolla, CA (US)

(21) Appl. No.: 16/646,956

(22) PCT Filed: Sep. 14, 2018
(86) PCT No.: PCT/US2018/051135

§ 371 Date: Mar. 12, 2020

Related U.S. Application Data
(60) Provisional application No. 62/558,667, filed on Sep. 14, 2017.

(51) Int. Cl. C12N 5/0789

(52) U.S. Cl. CPC C12N 5/0647; C12N 2510/00; C12N 2501/60

(57) ABSTRACT

Disclosed herein are methods for promoting the maintenance, expansion, and transplantation of stem cells by culturing the cells under conditions which increase expression and/or activity of Hsf1.

### RELATED APPLICATIONS

This application claims the benefit of U.S. Provisional Application 62/558,667, filed Sep. 14, 2017, the entire contents of which are incorporated by reference herein.

### BACKGROUND

Many tissues and organs contain stem cells. These stem cells have the capacity to self-renew (make more of themselves) and to differentiate into all of the specialized cell types within a given tissue or organ. As a consequence, stem cells have enormous therapeutic potential for treating diverse diseases by replacing cells, tissues or organs that have been damaged, lost or malfunction as a consequence of injury, disease or aging. Unfortunately, stem cells are very rare cells, and their abundance can be limiting for therapy. For example, many blood cancer patients require hematopoietic stem cell (HSC) transplants to replenish their blood forming system after chemotherapy. However, appropriate HSC donors are not always found, and thus many patients cannot receive HSC transplants because there is an insufficient source of stem cells available. One strategy to overcome this shortage is to grow and expand stem cells outside of the body in cell culture. A severe limitation for implementing this strategy is the technical difficulty in growing and expanding stem cells in culture. Strategies that enable stem cell maintenance and expansion must be developed to overcome these unmet clinical needs.

### SUMMARY

Herein disclosed are methods of maintaining and/or expanding mammalian stem cells in culture. In some embodiments these methods comprise culturing the stem cells in the presence of an enhancer of protein homeostasis capacity, a “stem cell protein homeostat (SCp homeostats). In some embodiments these methods comprise culturing the stem cells in the presence of an agent that increases Hsf1 expression and/or activity, an “Hsf1 activator”. In some embodiments the Hsf1 activator is a Heat shock transcription factor 1 (Hsf1) agonist. In some embodiments the Hsf1 activator is an HSP90 inhibitor, for example, 17-AAG (17-N-Allylamino-17-demethoxygeldanamycin), geldanamycin, radicicol, IPI-504, IPI-493, 17-DMAG, CNF-1010, macbecin, CCT018159, VER-49009, B11B021, AT-13387, PF-04928473, STA-9090, or AUY922. In some embodiments the Hsf1 activator is an inhibitor of the chaperonin TRiC, for example, HSF1A or 2-[(4-chloro-2λ4,1,3-benzothiadiazol-5-yl)oxy]acetic acid. In some embodiments combinations of two or more such agents (whether an Hsf1 activator or a SCp homeostat) are used. In some embodiments particular type(s) or species of agent are specifically included. In some embodiments particular type(s) or species of agent are specifically excluded.

Also disclosed are related methods comprising exposing stem cells to an agent that increases Hsf1 expression and/or activity as set out above. In some embodiments such methods include methods to collect or store mammalian stem cells. In some embodiments the storage is short-term. In other embodiments the storage is long-term.

In some embodiments the stem cells are cultured for more than seven days wherein the stem cells retain regenerative activity. In some embodiments the stem cells are cultured for at least 8, 9, 10, 11, 12, 13 or 14 days, wherein the stem cells retain regenerative activity. The stem cells can be cultured without co-culturing with stromal cells. In some embodiments the culture medium is serum-free or non-human serum free.

Some embodiments comprise exposing a stem cell to, or culturing a stem cell in the presence of, an HSP90 inhibitor, for example, 17-AAG. Some embodiments comprise exposing a stem cell to, or culturing a stem cell in the presence of, an inhibitor TRiC, for example, HSF1A.

In some embodiments such methods include potentiating the transplantation or implantation of stem cells in a subject in need thereof. In various aspects of the transplantation and implantation embodiments, the need is for regenerative therapy or for cell therapy. In further aspects of these embodiments the stem cells so treated or their progeny exhibit improved establishment or engraftment in the subject compared to stem cells or their progeny that have been cultured in the absence of an agent that increases Hsf1 expression and/or activity.

Conditions that can be treated by regenerative therapy include bone marrow failure syndromes, diabetes, spinal cord injury, muscular dystrophy, osteoarthritis, rheumatoid arthritis, bone fractures, joint injury or degeneration, cardiovascular disease, autoimmune disease, Parkinson's disease, anemia, HIV/AIDS, macular degeneration, thalassemias, amyotrophic lateral sclerosis, Huntington's disease, kidney failure, severe combined immunodeficiency, sickle cell disease.

Conditions that can be treated by cell therapy include solid cancers, blood cancers, inflammation, vascular disorders.

In some embodiments such methods include genetically modifying mammalian stem cells in culture or preparing stem cells for genetic modification. These methods comprise a step of obtaining, maintaining, and/or expanding the stem cells in media comprising an agent that increases Hsf1 expression and/or activity. Some embodiments further comprise exposing the stem cells to a gene therapy or genetic modification reagent. In some embodiments the gene therapy or genetic modification reagent is a viral vector, for example a lentivirus vector. In some embodiments the gene therapy or genetic modification reagent mediates insertion, deletion, or editing of the stem cell's DNA. In some embodiments the gene therapy or genetic modification reagent confers episomal expression of a polypeptide or non-translated RNA molecule. In some embodiments genetic modification is mediated by CRISPR, zinc-finger nuclease, or TALEN technology. Some embodiments further comprise transplanting the genetically modified stem cells, or their progeny in a subject in need thereof.

In some of the herein disclosed embodiments the mammalian stem cell is a multipotent stem cell. In some embodiments the multipotent stem cell is hematopoietic stem cell (HSC), a neural stem cell, a skin stem cell, a muscle stem cell, a germ-line stem cell, a mesenchymal stem cell, a skeletal stem cell, a pancreatic stem cell, a liver stem cell, a cardiac stem cell, a hair follicle stem cell, an endothelial stem cell, an epithelial stem cell, a mammary stem cell, an adipose-derived stem cell, or an intestinal stem cell. In some of the herein disclosed embodiments the stem cell is isolated from a donor, from the indicated tissue. In some embodiments HSC are isolated from umbilical cord blood, bone marrow, or mobilized peripheral blood. In various aspects the donor is autologous or allogeneic. In some of the herein disclosed embodiments the stem cell is derived from a pluripotent stem cell, such as an induced pluripotent stem cell (iPSC) or an embryonic stem cell. Some embodiments specifically include stem cells of a particular type or source. Other embodiments specifically exclude stem cells of a particular type or source.

Some embodiments are methods of screening one or more drug candidates for activity on mammalian stem cells or progenitor cells comprising culturing cells obtained from culturing stem cells (such as stem cells or progenitor cells) in the presence of an agent that increases Hsf1 expression and/or activity with the drug candidate(s).

Further embodiments are combinations of reagents or kits for practicing any of the herein disclosed methods comprising one or more Hsf1 activators or SCp homeostats. Such kits may further comprise a culture medium. Such kits can further comprise instructions for culturing, maintaining and expanding the stem cells. In some embodiments the medium is serum-free or non-human serum free.

Some embodiments are pharmaceutical compositions comprising stem cells or their progeny that have been cultured or stored in the presence of an Hsf1 activator or SCp homeostat and a pharmaceutically acceptable carrier. Pharmaceutically acceptable carriers for cellular product are typically sterile aqueous solutions. In various aspects of these embodiments the pharmaceutically acceptable carrier can comprise culture media, phosphate-buffered saline, or HEPES-buffered saline. In some embodiments the media is serum-free or non-human serum free.

Throughout this disclosure many embodiments are described making reference to HSC and Hsf1 activators (agents that increase Hsf1 expression and/or activity). For each such embodiment there are parallel embodiments in which the Hsf1 activator is specifically an HSP90 inhibitor or a TRiC inhibitor or a subgenus or individual species thereof. Similarly, there are parallel embodiments in which a SCp homeostat, or a subgenus or individual species thereof, is used instead of an Hsf1 activator. There are also further parallel embodiments relating to multipotent stem cells other than HSC, multipotent stem cells generally, and to subgenera thereof.

In several of the herein disclosed embodiments, mammalian multipotent stem cells or their progeny are prepared for administration to a mammal or administered to a mammal. In some embodiments the mammal is a human. In other embodiments the mammal is a domestic pet, for example a cat or a dog. In some embodiments the mammal is an agricultural animal, for example, a horse, a cow, a sheep, or a hog. In other embodiments, the mammal is a laboratory animal, for example a mouse, a rat, a hamster, or a rabbit.

### BRIEF DESCRIPTION OF THE DRAWINGS

FIG. 1A-D depict quantification of protein synthesis in hematopoietic cells. FIG. 1A depicts how 0-propargyl-puromycin (OPP) was used to quantify protein synthesis within cells. OPP is a puromycin analog with a terminal alkyne group that mimics an aminoacyl-tRNA and integrates into nascent polypeptides. Azide-alkyne cycloaddition can covalently link a fluorescently tagged azide molecule to OPP that has been incorporated into new peptides. FIG. 1B depicts that when OPP was added to cell culture medium, its incorporation in primary bone marrow cells was detected and quantified by flow cytometry. The incorporation of OPP was inhibited by pre-treatment with the translation inhibitor cycloheximide (CHX), indicating that OPP incorporation is a measure of new protein synthesis. FIG. 1C depicts OPP incorporation in CD150⁺CD48⁻Lineage⁻Sca1⁺ckit⁺ (CD150⁺CD48⁻LSK) HSCs and unfractionated bone marrow cells one hour after administration in vivo. FIG. 1D depicts OPP incorporation (protein synthesis rates) in HSCs, multipotent progenitors (MPP), common myeloid progenitors (CMP), granulocyte macrophage progenitors (GMP), granulocytes (GR) megakaryocyte erythroid progenitors (MEP), CD71⁺Ter119⁺ erythroid progenitors (E), pro-B, pre-B, IgM⁺ B cells (IgM), CD3⁺ T cells (T), and unfractionated bone marrow cells (BM). HSCs exhibited lower rates of protein synthesis than almost all other hematopoietic cells. Statistical significance relative to HSCs was assessed using a repeated-measures one way ANOVA followed by Dunnett's test (n=9; **P<0.01, ***P<0.001).

FIG. 2A-B depict that HSCs require a highly regulated protein synthesis rate. FIG. 2A depicts OPP incorporation into HSCs of the indicated genotypes (n=15). A mutation in the ribosomal gene Rp124 (Rp124^{Bst/+}) reduced protein synthesis by about 40% as compared to wild-type HSCs. Deletion of the gene Pten increased protein synthesis by about 30% as compared to wild-type HSCs. Rpl24^{Bst/+} blocked the increase in protein synthesis that normally occurred when Pten was deleted. FIG. 2B depicts donor cell engraftment when 10 HSCs were transplanted with 3×10⁵ recipient-type (wild-type, congenic) bone marrow cells into irradiated recipients (n=12-19). Statistical significance was assessed with a one way ANOVA followed by Dunnett's test. Significance relative to wild-type (+/+; ## P<0.01, ### P<0.001) or Pten^{-/-} (*P<0.05, **P<0.01, ***P<0.001).

FIG. 3 depicts the protein homeostasis network. Protein homeostasis can be regulated by translation, protein folding (mediated by chaperones) and protein degradation. Mistranslation and improper folding can lead to an accumulation of misfolded proteins that can malfunction or form toxic aggregates. Misfolded proteins can induce a proteotoxic stress response, which is controlled by Hsf1.

FIG. 4A-C depict that HSCs have high protein quality. FIG. 4A depicts a western blot of 3×10⁴ cells from each cell population showing that ubiquitylated protein levels are lower in CD48⁻LSK HSCs/MPPs as compared to more differentiated progenitor cells. The ladder/smear of protein shows the accumulation of polyubiquitylated proteins, which predominantly marks misfolded and defective proteins. FIG. 4B depicts cell volume based on diameter and assuming spherical shape (n>60). FIG. 4C depicts total protein isolated from 5×10⁴ cells of each population measured by micro-BCA assay (n=3). Statistical significance relative to HSC/MPP was assessed with a one way ANOVA followed by Dunnett's test (*P<0.05, **P<0.01, ***P<0.001).

FIG. 5 depicts that HSCs do not have high proteasome activity. Proteasome activity was measured in 3×10⁴ HSCs/MPPs, CMPs, GMPs, and MEPs. HSCs treated with the proteasome inhibitor MG132 were used as a control. Statistical significance relative to HSCs/MPPs was assessed with a one way ANOVA followed by Dunnett's test (n=3; *P<0.05, **P<0.01).

FIG. 6A-D depict the validation of tetraphenylethene maleimide (TMI) as a probe to measure unfolded protein. FIG. 6A depicts that TMI is a cell permeable dye that fluoresces upon binding to cysteine thiols. The free thiol side chains of non-disulphide bonded hydrophobic cysteines are typically buried within the core of globular proteins, but can be exposed in unfolded proteins. FIG. 6B depicts that TMI fluorescence was higher in bone marrow cells after a 4 hour incubation at 42° C. as compared to 37° C. Since heat shock induces protein unfolding, this validated TMI as a means to quantify unfolded protein. FIG. 6C depicts that TMI fluorescence in bone marrow cells was increased after treatment with the proteasome inhibitor bortezomib. FIG. 6D depicts a western blot demonstrating that bone marrow cells with high TMI fluorescence contain more ubiquitylated protein than bone marrow cells with low TMI fluorescence. FIG. 6B-D validated that TMI can be used to quantify unfolded protein (protein quality) in hematopoietic cells.

FIG. 7 depicts that HSCs have low abundance of unfolded protein. TMI fluorescence (unfolded protein abundance) was quantified in HSCs, MPPs, CMPs, GMPs, and MEPs. HSCs had significantly less unfolded protein than each of these more differentiated progenitor cell populations. Statistical significance relative to HSCs was assessed with a one way ANOVA followed by Dunnett's test (n=11; *P<0.05, **P<0.01).

FIG. 8A-B depict that increasing protein synthesis rates reduces protein quality within HSCs. FIG. 8A depicts a western blot of 3×10⁴ CD48⁻LSK HSCs/MPPs of the indicated genotypes. FIG. 8B depicts TMI fluorescence (unfolded protein abundance) within HSCs of the indicated genotypes. Pten-deficiency increases protein synthesis rates (FIG. 2A), ubiquitylated and unfolded protein abundance (FIG. 8). Each of these changes is blocked by Rpl24^{Bst/+}.

FIG. 9A-D depict that Aars^{sti/sti} HSCs accumulate ubiquitylated protein and have impaired maintenance and function. FIG. 9A depicts a western blot that examined ubiquitylated protein levels in 2.5×10⁴ CD48-LSK HSCs/MPPs, CMPs, GMPs, and MEPs isolated from Aars^{sti/sti} (sti/sti) or littermate control (+/+) mice. FIG. 9B depicts that the frequency of HSCs is reduced in Aars^{sti/sti} (sti/sti) bone marrow as compared to controls (+/+) (n=3). FIG. 9C depicts that donor cell engraftment after secondary transplantation of bone marrow cells from Aars^{sti/sti} (sti/sti) is reduced as compared to controls (+/+) (n=20-23). FIG. 9D depicts that fewer secondary transplant recipients of Aars^{sti/sti} (sti/sti) bone marrow cells exhibited long-term (16-week) multilineage reconstitution of the blood system as compared to recipients of wild-type control cells. Statistical significance was assessed with a two-tailed Student's t-test (*P<0.05; ***P<0.001).

FIG. 10A-C depicts increased protein synthesis and reduced protein quality within HSCs in vitro. FIG. 10A depicts relative protein synthesis rates of HSCs normalized to bone marrow cells in vivo (n=6) and after 4 hours in vitro (n=5). Statistical significance was assessed with a two-tailed Student's t-test (***P<0.001). FIGS. 10B and 10C depict western blots that examined ubiquitylated protein levels in freshly isolated or cultured CD48⁻LSK HSCs/MPPs. Ubiquitylated protein significantly accumulated within 4 hours in vitro (FIG. 10B) and massively accumulated within 18 hours (FIG. 10C) in vitro. The accumulation of ubiquitylated protein was prevented by addition of the translation inhibitor cycloheximide (CHX) to culture medium.

FIG. 11 depicts our model that addition of an HSP90 inhibitor (e.g. 17-AAG), a TRiC inhibitor (e.g. HSF1A), an agonist of Hsf1 expression/activity or an enhancer of protein homeostasis capacity to culture medium can induce Hsf1 accumulation within HSCs in vitro and promote stem cell maintenance and expansion.

FIG. 12A-B depict that Hsf1 deletion does not impair HSC maintenance in vivo. FIG. 12A depicts a western blot that demonstrated that Hsf1 is efficiently deleted in Mx1-Cre⁺; Hsf1^{fl/fl} HSCs 1 week after pI:pC treatment. FIG. 12B depicts that donor cell engraftment in the peripheral blood is similar in recipients of 5×10⁵ Hsf1-deficient or control bone marrow cells 16 weeks after transplantation with 5×10⁵ wild-type congenic bone marrow cells (n=4-5).

FIG. 13A-B depict that Hsf1 deletion impairs HSC maintenance and protein homeostasis in vitro. FIG. 13A depicts donor cell engraftment in the peripheral blood 16 weeks after transplantation of 10 cultured (10 day) Hsf1-deficient or control HSCs with 2×10⁵ wild-type congenic bone marrow cells into irradiated recipients (n=5). FIG. 13B depicts a western blot that showed an accumulation of ubiquitylated protein in 5×10³ Hsf1-deficient (−/−) HSCs/MPPs as compared to controls (+/+) after 18 hours in vitro. Statistical significance was calculated with a two-tailed Student's t-test (*P<0.05, **P<0.01).

FIG. 14 depicts representative immunofluorescence staining of HSCs cultured for 10 days in the presence or absence of 17-AAG or HSF1A. Addition of 17-AAG or HSF1A to culture medium increased Hsf1 accumulation in the nucleus of cultured HSCs. Hsf1-deficient HSCs and cells stained without primary antibody (Neg Con) are shown as controls.

FIG. 15 depicts that addition of 17-AAG to culture medium reduced TMI fluorescence (unfolded protein abundance) within cultured HSCs (18 hour cultures shown).

FIG. 16A-B depict that HSC functionality is maintained and expanded in vitro by interventions that can induce/activate Hsf1. FIG. 16A depicts donor cell engraftment 16 weeks after transplantation of 10 HSCs cultured for 10 days in the presence or absence of an HSP90 inhibitor (17-AAG) or TRiC inhibitor (HSF1A) or from 10 freshly isolated HSCs. Cells were transplanted with 2×10⁵ freshly isolated congenic bone marrow cells (n=3 independent experiments with 3-5 recipients/condition/experiment). FIG. 16B depicts the frequency of transplant recipients that exhibited long-term (16 week) multilineage reconstitution (>0.5% B-, T-, and myeloid cell reconstitution) in primary (top) and secondary (bottom) transplants (n=13-18; Relative to untreated *P<0.05, **P<0.01, ***P<0.001; Relative to fresh # P<0.05, ## P<0.01, ### P<0.001). Addition of either 17-AAG or HSF1A increased the reconstituting potential of cultured HSCs as compared to both control cultured and freshly isolated HSCs.

FIG. 17 depicts that Hsf1 is required for 17-AAG and HSF1A to enhance HSC maintenance and expansion in vitro. Donor cell engraftment 12 weeks after transplantation of 10 wild-type and Hsf1-deficient HSCs cultured for 10 days in the presence or absence of 17-AAG, HSF1A or DMSO. Cells were transplanted with 2×10⁵ freshly isolated congenic bone marrow cells (n=7-9; *P<0.05, **P<0.01).

FIG. 18 depicts that addition of 17-AAG to culture medium increased the frequency of CD150⁺CD48⁻LSK HSCs in 10-day HSC cultures as compared to controls.

FIG. 19 depicts that addition of 17-AAG to culture medium supported highly efficient genetic modification of HSCs. The frequency of HSCs that were transduced with a GFP encoding retrovirus in culture is shown.

### DETAILED DESCRIPTION

Hematopoietic stem cell (HSC) transplants are used to treat patients with a broad spectrum of hematological malignancies, immune disorders and genetic blood diseases. Unfortunately, even after decades of use and research, there is a significant shortage of HSCs available for transplants. The majority of HSCs are derived from bone marrow or mobilized peripheral blood, but histocompatible donors are often lacking. Transplantable HSCs can also be derived from umbilical cord blood or pluripotent stem cells, but are typically too few in number to successfully transplant an adult patient. One approach to overcome this challenge would be to develop a means to maintain, grow and expand HSCs in vitro. Unfortunately, even after decades of research, there is no well-defined reproducible means to maintain or expand HSCs in vitro. Even short culture times in optimized conditions are deleterious to HSCs. Ex vivo HSC maintenance and expansion could significantly enhance their clinical utility in a wide range of human diseases (e.g. various leukemias, anemias, hemoglobinopathies, inherited immune system disorders, inherited metabolic disorders, HIV/AIDS etc.), provide a new platform for testing drugs within stem cells, enable efficient genetic modification of stem cells, and develop into a widely used tool for the research community.

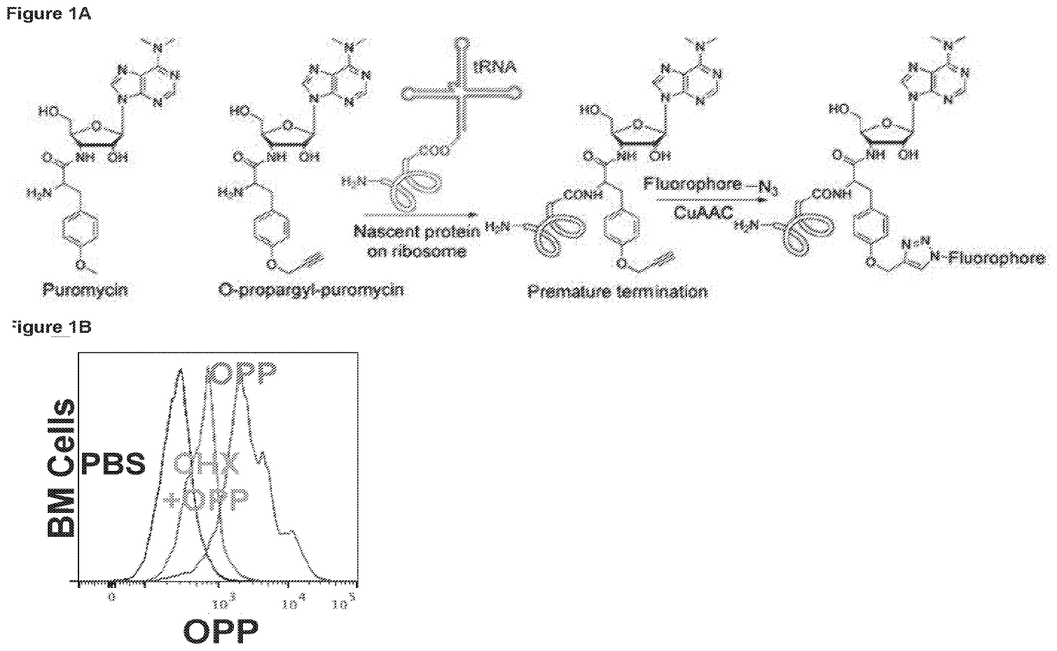

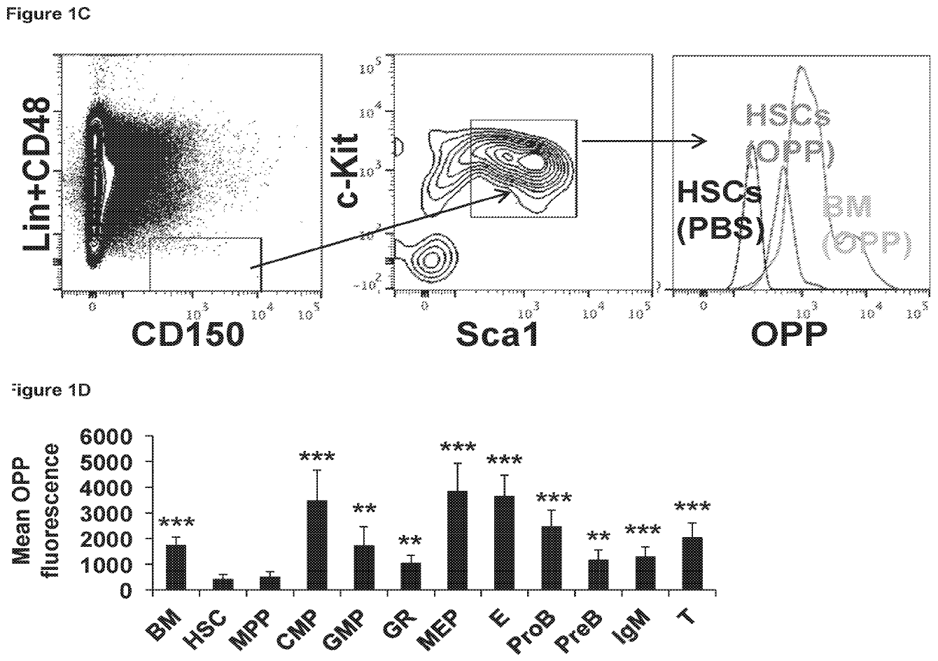

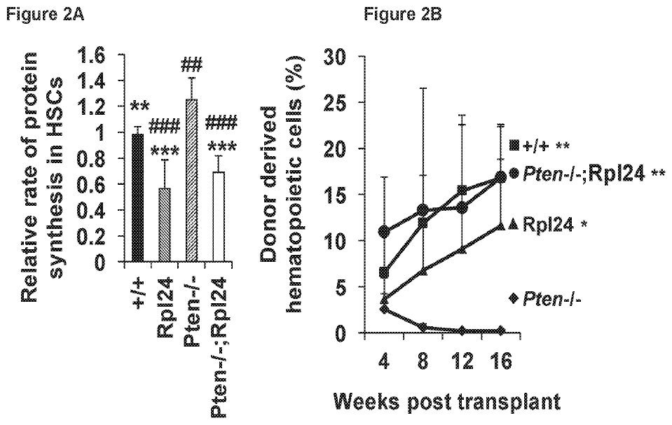

We recently observed that HSCs have lower rates of protein synthesis than other blood cells (FIG. 1). Low protein synthesis is necessary for HSCs, as genetic changes that increase protein synthesis impair HSC function (FIG. 2). Stem cells in other tissues also have relatively low rates of protein synthesis, for example, neural stem cells, skin stem cells, muscle stem cells, germ-line stem cells, and intestinal stem cells. It has now been discovered that this low rate of protein synthesis is not exhibited under typical culture conditions but is generally necessary for stem cell maintenance. Why low protein synthesis is necessary for stem cells and how it contributes to their maintenance and function has not been previously addressed.

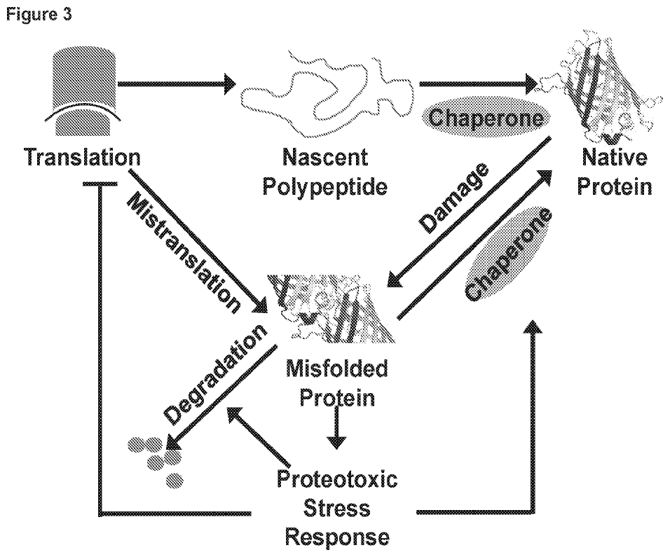

Protein synthesis can be highly error prone. High rates of protein synthesis can increase amino acid misincorporation. Translational errors can lead to protein misfolding and the formation of toxic aggregates (FIG. 3). It is herein disclosed that low protein synthesis promotes HSC maintenance by preventing the synthesis of defective/misfolded proteins, thereby enhancing protein quality and homeostasis.

As used herein, the term “stem cells” refer to cells with the ability to both replace themselves and to differentiate into more specialized cells. Their self-renewal capacity generally endures for the life-span of the organism. A pluripotent stem cell can give rise to all the various cell types of the body. A multipotent stem cell can give rise to a limited subset of cell types. For example, a hematopoietic stem cell can give rise to the various types of cells found in blood, but not to other types of cells. Multipotent stem cells can also be referred to as somatic stem cells, tissue stem cells, lineage-specific stem cells, and adult stem cells. The non-stem cell progeny of multipotent stem cells are progenitor cells (also referred to as restricted-progenitor cells). Progenitor cells give rise to fully differentiated cells, but a more restricted set of cell types than stem cells. Progenitor cells also have comparatively limited self-renewal capacity; as they divide and differentiate they are eventually exhausted and replaced by new progenitor cells derived from their upstream multipotent stem cell.

As used herein, the phrase “maintaining stem cells” refers not just to culturing the stem cells in a manner preserving their viability, but also to retaining their functionality as stem cells, that is, to being self-renewing and capable of giving rise to the full range of progenitor lineages appropriate to the particular type of stem cell (these two functions together “regenerative activity”). One way of demonstrating that stem cells have been successfully maintained is through an engraftment experiment in which all the appropriate cell types (bearing a genetic marker distinguishing them from the host) are observed to arise from the graft and remain present over an extended period of time, for example 4 months.

As used herein, the phrase “expanding stem cells” refers not just to maintaining the stem cells but to culturing the stem cells in a manner that the number of stem cells in the culture increases. One way of demonstrating that stem cells have been successfully expanded is an engraftment experiment comparing the percentage of donor-derived cells obtained from transplants of cultured and freshly isolated stem cells. The comparison is based on transplanting the same number of freshly isolated stem cells as were originally placed in culture. An increased percentage of donor-derived cells in the recipients of the cultured stem cells as compared to in the recipients of the freshly-isolated stem cells is consistent with the successful expansion of the stem cells in culture.

As used herein, the phrase “protein homeostasis capacity” refers to the ability/capacity of a cell to maintain normal levels of misfolded, unfolded, ubiquitylated, and defective proteins under varying conditions. Multipotent stem cells exhibit a limited protein homeostasis capacity and experience a dramatic rise in the accumulation of misfolded, unfolded ubiquitylated, or defective proteins when cultured in vitro. By enhancing protein homeostasis capacity, through use of a stem cell protein homeostat normal stem cell function can be maintained. Is some embodiments Hsf1 activators are SCp homeostats. In some embodiments the SCp homeostat is celasterol.

In some embodiments, the SCp homeostat can include an mTor inhibitor, while in other embodiments the SCp homeostat specifically excludes an mTor inhibitor. In some aspects of these embodiments the mTor inhibitor is rapamycin. In some embodiments, the SCp homeostat can include a GSK3 inhibitor while in other embodiments SCp homeostat specifically excludes a GSK3 inhibitor. In some aspects of these embodiments the GSK3 inhibitor is lithium. In some embodiments the SCp homeostat can include an aryl hydrocarbon receptor antagonist while in other embodiments SCp homeostat specifically excludes an aryl hydrocarbon receptor antagonist. In some aspects of these embodiments the aryl hydrocarbon receptor antagonist is StemRegenin 1. In some embodiments, these exclusions do not extend to the Hsf1 activators and SCp homeostats disclosed herein, even if there is overlapping activity with the genera and species excluded in this paragraph.

Still in further embodiments, the Hsf1 activators and SCp homeostats specifically exclude valporic acid, or a pyrimidoindole derivatives, such as UM171.

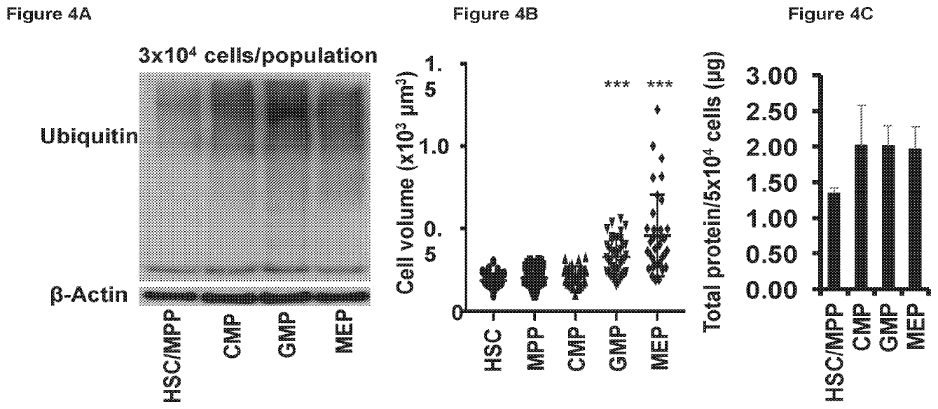

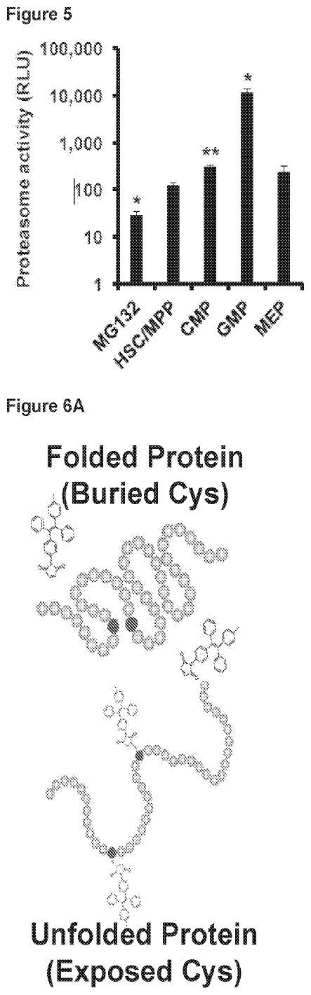

It has been determined that HSCs contained less ubiquitylated protein than restricted progenitors (FIG. 4A). Ubiquitylated protein is a surrogate widely used to quantify misfolded proteins. Low levels of ubiquitylated protein within HSCs are not explained by differences in cell volume (FIG. 4B), total protein abundance (FIG. 4C) or proteasome activity (FIG. 5).

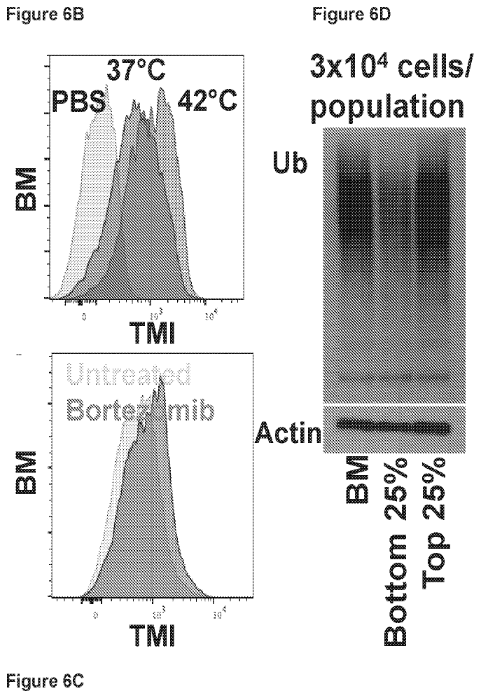

As a complement to evaluating protein quality by assessing changes in ubiquitylated protein, a quantitative orthogonal assay to measure protein quality was developed. Tetraphenylethene maleimide (TMI) is a cell permeable dye that fluoresces upon binding to cysteine thiols. The free thiol side chains of non-disulphide bonded hydrophobic cysteines are typically buried within the core of globular proteins, but can be exposed in unfolded proteins (FIG. 6A). We adapted TMI to quantify unfolded proteins within single hematopoietic cells by flow cytometry. We validated TMI by assessing the effect of heat shock, which induces protein unfolding, on TMI fluorescence in bone marrow cells. Bone marrow cells incubated at 42° C. for 4 hours exhibited a significant increase in TMI fluorescence relative to an aliquot of the same cells incubated at 37° C. (FIG. 6B). In addition, we compared levels of ubiquitylated protein within TMI^{low} (lowest quartile of TMI fluorescence), TMI^{high} (highest quartile of TMI fluorescence) and unfractionated bone marrow cells by western blot. TMI^{low} bone marrow cells contained less ubiquitylated protein than unfractionated bone marrow cells, which in turn contained less ubiquitylated protein than TMI^{high} bone marrow cells (FIG. 6D). Finally, since unfolded proteins are primarily degraded by the ubiquitin proteasome system, we assessed the effects of proteasome inhibition on TMI fluorescence in vivo. Bone marrow cells isolated from mice treated with the proteasome inhibitor Bortezomib exhibited a ˜30% increase in TMI fluorescence as compared to cells from vehicle treated controls (FIG. 6C). These experiments indicate that TMI is an effective tool for measuring unfolded protein abundance within hematopoietic cells.

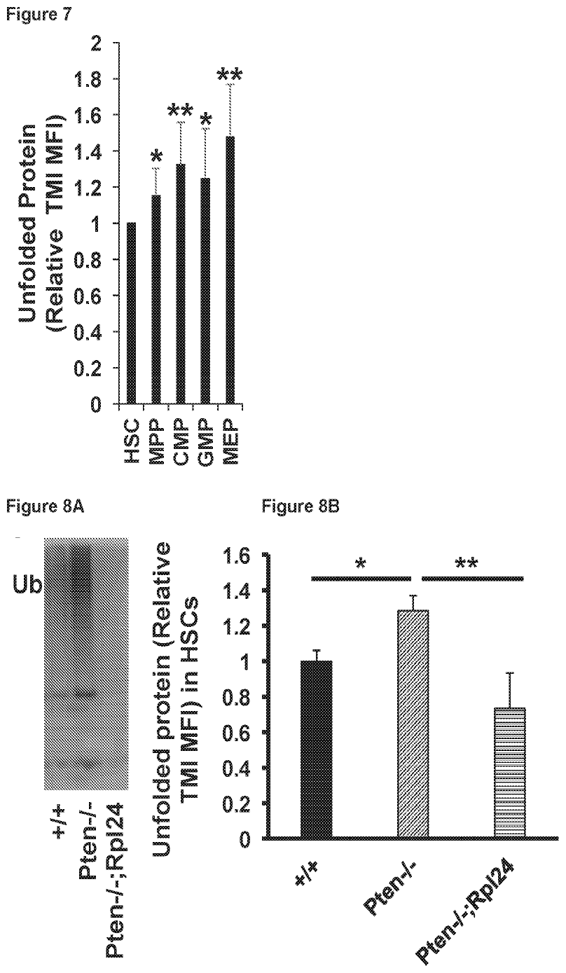

Using TMI, it was determined that HSCs contained significantly less unfolded protein as compared to more differentiated progenitors (FIG. 7). Taken together with the data on ubiquitylated protein, these findings indicate that stem cells have high protein quality and elevated protein homeostasis capacity as compared to several more differentiated cell types.

Modest increases in protein synthesis led to increased ubiquitylated and unfolded protein (FIG. 8). Pten-deficient HSCs exhibited a ˜30% increase in their rate of protein synthesis (FIG. 2A). This was accompanied by increased ubiquitylated and unfolded protein (˜30%) (FIG. 8). A mutation in the ribosomal gene Rp124 blocked the increase in protein synthesis within HSCs after Pten deletion (FIG. 2A), and also blocked the accumulation of ubiquitylated and unfolded protein (FIG. 8). These data indicate that increasing protein synthesis reduces protein quality within HSCs.

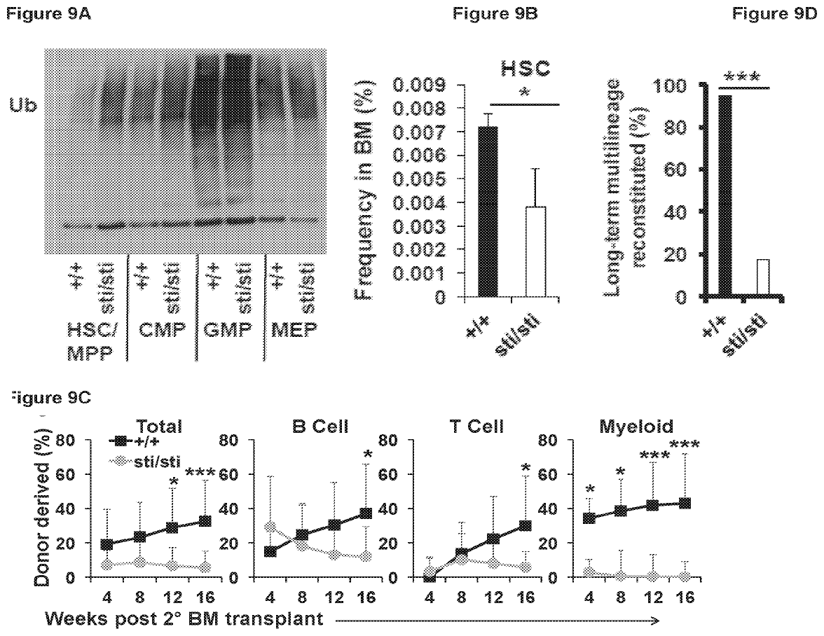

The relationship between protein synthesis and protein quality in HSCs indicated that declines in protein quality or imbalanced protein homeostasis impaired HSCs. To demonstrate this, the properties of HSCs from Aars^{sti/sti} mice, that harbor a mutation in the alanyl-tRNA synthetase that causes a tRNA editing defect, were examined. This mutation increases the error rate during protein synthesis, which can lead to an accumulation of misfolded proteins and diminished protein homeostasis capacity. Aars^{sti/sti} HSCs exhibited increased ubiquitylated protein (FIG. 9A), which indicate that protein homeostasis was perturbed in these cells. The abundance of HSCs was reduced in Aars^{sti/sti} bone marrow (FIG. 9B). Aars^{sti/sti} HSCs also exhibited impaired regenerative function and self-renewal potential. Aars^{sti/sti} HSCs exhibited a significant reduction in their capacity to regenerate the blood upon serial transplantation in irradiated recipients (FIG. 9C, 9D). These data indicate that modest declines in protein quality, assessable as abundance of unfolded, misfolded and/or ubiquitylated protein, impair stem cell maintenance (e.g., viability) and function (e.g., transplantability).

Strikingly, it was discovered that HSCs rapidly increased their rate of protein synthesis in vitro (FIG. 10A), and this was associated with a massive increase in ubiquitylated protein (FIG. 10B, 10C). The accumulation of ubiquitylated protein could be blocked by adding the translation inhibitor cycloheximide to the culture medium (FIG. 10B, 10C). These data indicate that protein synthesis is dramatically increased within HSCs in vitro, and that this causes a decline in protein quality and homeostasis. Based on all of these data, it was concluded that increased protein synthesis and imbalanced protein homeostasis impair HSC self-renewal and contribute to HSC depletion in vitro.

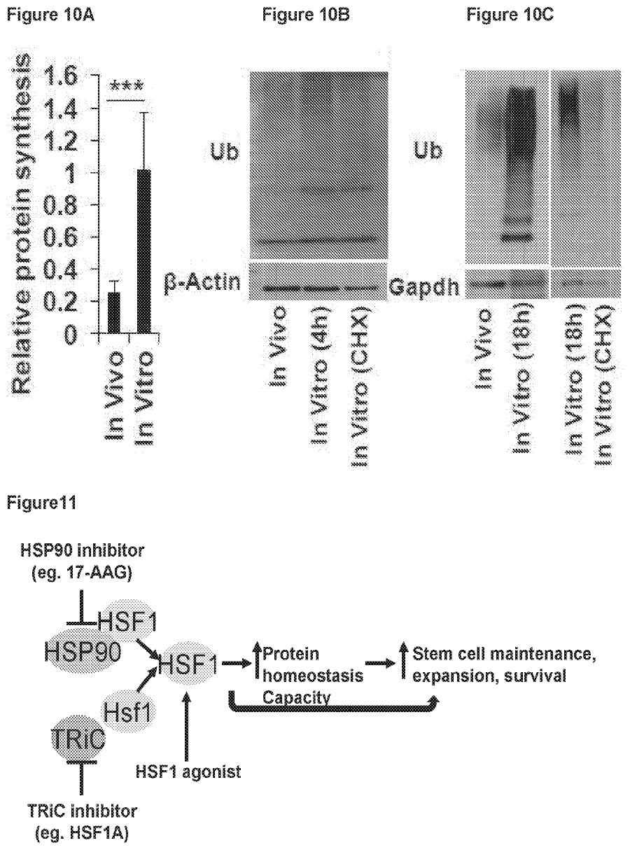

The main cellular response to proteotoxic stress is activation of the heat shock pathway. The master regulator of this pathway in eukaryotes is Heat shock factor 1 (Hsf1), which encodes a highly conserved transcription factor that promotes protein homeostasis. At steady state, inactive Hsf1 is typically localized in the cytoplasm where it binds to chaperones/chaperonins, such as HSP90 and TRiC (FIG. 11). When Hsf1 is unencumbered, it can translocate to the nucleus where it can induce a genetic program to enhance protein homeostasis.

It is the non-binding hypothesis of the present inventor that increasing Hsf1 expression and/or activity drives a gene expression program that promotes stem cell maintenance, expansion and survival (FIG. 11). Empirically, agents such as HSP90 inhibitors and TRiC inhibitors do promote maintenance, expansion and survival of stem cells, and thus each of the herein disclosed embodiments may make use of such reagents without reference to any particular mechanism. Moreover, Hsf1 expression is necessary for the effect of these agents. It is the inventor's current understanding that these agents, increase Hsf1 activity (by increasing Hsf1 expression, or otherwise) and that Hsf1 acts to increase protein homeostasis capacity in the stem cells. As the accumulation of misfolded, unfolded, or ubiquitylated proteins is correlated with the loss of stem cell functionality, other agents that increase protein homeostasis capacity including inhibitors of protein synthesis, for example, cycloheximide, hippuristinol and silvestrol, and activators or autophagy, such as, rapamycin, vampric acid, lithium, SMER28, and trehalose can also be useful for promoting the maintenance, expansion and survival of stem cells. Such agents may be referred to as stem cell protein homeostats (SCp homeostats). Some embodiments specifically include one or more of these various subgenera or species. Some embodiments specifically exclude one or more of these various subgenera or species.

Thus, disclosed herein are methods of maintaining and expanding HSCs in culture in the presence of compounds or factors which increase Hsf1 expression and/or activity. Non-limiting examples of such compounds are HSP90 inhibitors (such as 17-AAG), TRiC inhibitors (such as HSF1A), and other Hsf1 activators. As a result of such processes the stem cells retain regenerative activity, that is, the capacity for self-renewal and the capacity to give rise to the full complement of progenitor lineages.

The concept of using an Hsf1 activator to maintain stem cells in culture can be applied more broadly beyond simply culturing the stem cells. In some situations stem cells may not be cultured at all, but stored between isolation and transplantation (or stored after culturing). Thus in some embodiments an Hsf1 activator is added to the storage medium. For short term storage of a few hours to a few days at room temperature or refrigerated (e.g. 4° C.), the storage medium can be, for example, culture medium, phosphate-buffered saline, or HEPES-buffered saline. For longer term storage, the stem cells would be cryogenically frozen and the Hsf1 activator would be added to the cryogenic storage medium, which can comprise, for example, glycerol, DMSO, or serum. By storing the stem cells in a medium containing an Hsf1 activator they can have better viability in culture and better transplantability in comparison with stem cells stored without use of an Hsf1 activator. In some embodiments the medium is serum-free or non-human serum free.

The culturing of stem cells in many instances would be undertaken as a preparative step for their eventual transplantation. Thus some embodiments are methods of preparing stem cells for transplantation by culturing them in the presence of an Hsf1 activator. Similarly, culturing stem cells in the presence of an Hsf1 activator can promote their expansion. Thus some embodiments are methods of potentiating the transplantation or engraftment of the stem cells. By potentiation it is meant that a greater or more rapid effect is achieved than if the Hsf1 activator were not used. Thus by promoting the expansion of the stem cells, more stem cells are available and used for the transplantation procedure leading to a higher level of engraftment in for example a regenerative therapy or a quicker or greater effect in a cellular therapy. As used herein “regenerative therapy” refers to the use of stem cells or their progeny to replace or augment missing, depleted, or damaged tissue(s). An example of such use is the reconstitution of hematopoiesis following radiation therapy or high-dose chemotherapy for cancer. Other examples include the reconstitution of hematopoiesis for the treatment of thalassemia, sickle cell disease, aplastic anemia, autoimmune diseases, and immune deficiency syndromes. Other examples include the treatment of diabetes, heart failure, amyotrophic lateral sclerosis, Parkinson's disease, kidney failure, and macular degeneration. As used herein “cellular therapy” refers to the use of stem cells or their progeny as a therapeutic agent. An example of a cellular therapy is CAR-T cells, the production of which can be facilitated by the use of stem cell technology. Still further related embodiments are methods of cellular transplantation comprising administering stem cells or their progeny that have been cultured in the presence of an Hsf1 activator. Examples of this include using T cells, insulin producing cells, or neural cells for the treatment of cancer, diabetes, and spinal cord injury, respectively. In aspects of these embodiments the stem cells or their progeny have improved establishment or engraftment in a recipient compared to those cultured in the absence of an Hsf1 activator.

In some uses, for example in cellular therapies, the stem cells (or their progeny) can be genetically modified to express an exogenous or recombinant protein, or to alter the expression profile of an endogenous protein, to correct an inborn genetic mutation, or to express a protein ectopically. Thus some embodiments of culturing stem cells in the presence of an Hsf1 activator comprise an additional step of genetically modifying them. Many techniques for genetic modification are known to those of skill in the art including various ways of delivering nucleic acids or nucleic acid modifying enzymes into cells including physical (e.g., electroporation, sonoporation, particle bombardment), chemical transfection (e.g. cationic liposomes), and biologic (e.g., viral vectors). A transfected nucleic acid may directly express a therapeutic polypeptide or RNA, or it may encode components for modifying the host cell genome, such as zinc-finger nucleases, or components of TALEN or CRISPR technology, as are known in the art.

One impediment encountered in the development of stem cell based therapies has been an inability to reliably and reproducibly generate multipotent stem cells from pluripotent stem cells (e.g., induced pluripotent stem cells; iPSC). In light of the present disclosure, it is apparent that typical culture conditions are not suited to the maintenance and expansion of multipotent stem cells. Thus some embodiments disclosed herein are methods of deriving multipotent stem cells from pluripotent stem cells comprising culturing pluripotent stem cells in the presence of an Hsf1 activator or SCp homeostat, and differentiation factors promoting differentiation into particular multipotent stem cell lineages. Such differentiation factors are known in the art, and include cytokines (such as interleukin (IL) 3, IL-6, IL-11), growth factors (such as fibroblast growth factor (FGF) 3, FGF-10, FGF-7), bone morphogenic proteins (BMP) (such as BMP-2 and BMP-4), Wnt signaling molecules (such as Wnt-2a, Wnt-2b, Wnt-3a), and stem cell factor (SCF/ckit-ligand). Other embodiments include methods of deriving multipotent stem cells from pluripotent stem cells comprising differentiating the pluripotent stem cells in the presence of an Hsf1 activator or SCp homeostat.

Similarly, in some instances it will be desired to generate progenitor cells in vitro. Thus some embodiments are methods of deriving progenitor cells from multipotent stem cells comprising culturing multipotent stem cells in the presence of an Hsf1 activator and differentiation factors promoting differentiation into particular progenitor cell lineages. Such differentiation factors are known in the art. Examples include vascular endothelia growth factor (VEGF), thrombopoietin (TPO), erythropoietin (EPO), IL-7, insulin, keratinocyte growth factor (KGF), and collagen.

Some of the herein disclosed embodiments are methods of storing, culturing, maintaining, expanding, and preparing stem cells (and their progeny) practiced wholly extracorporeally. Other embodiments comprise administration to a mammal, for example a human, and constitute method of treatment. As used herein, the term “treating” or “treatment” broadly includes, both collectively and as individual embodiments, any kind of treatment activity, including the diagnosis, mitigation, or prevention of disease in man or other animals, or any activity that otherwise affects the structure or any function of the body of man or other animals. Treatment activity includes the administration of the medicaments, dosage forms, and pharmaceutical compositions described herein to a patient, especially according to the various methods of treatment disclosed herein, whether by a healthcare professional, the patient his/herself, or any other person. Treatment activities include the orders, instructions, and advice of healthcare professionals such as physicians, physician's assistants, nurse practitioners, and the like that are then acted upon by any other person including other healthcare professionals or the patient his/herself. In some embodiments, treatment activity can also include encouraging, inducing, or mandating that a particular medicament, or combination thereof, be chosen for treatment of a condition—and the medicament is actually used—by approving insurance coverage for the medicament, denying coverage for an alternative medicament, including the medicament on, or excluding an alternative medicament, from a drug formulary, or offering a financial incentive to use the medicament, as might be done by an insurance company or a pharmacy benefits management company, and the like. In some embodiments, treatment activity can also include encouraging, inducing, or mandating that a particular medicament be chosen for treatment of a condition—and the medicament is actually used—by a policy or practice standard as might be established by a hospital, clinic, health maintenance organization, medical practice or physicians group, and the like.

Administration of stem cells or their progeny is typically by injection or infusion. In some embodiments intravenous administration is used. In other embodiments the stem cells or their progeny are administered into a tissue, organ, or body cavity that is, or is in communication with, the site where treatment is to take effect.

Each method of treatment may be expressed as a composition(s) for use in such a medical method. For example, embodiments comprising an Hsf1 activator or an SCp homeostat for use in potentiating transplantation of stem cells or their progeny. Other embodiments include stem cells or their progeny cultured in the presence of an Hsf1 activator or an SCp homeostat for use in regenerative medicine, cell therapy, of cellular transplantation. Similarly, each method of treatment may be expressed as a composition(s) for use in the manufacture of a medicament. For example, embodiments comprising an Hsf1 activator or an SCp homeostat for use in the manufacture of a medicament for potentiating transplantation of stem cells or their progeny. Other embodiments include use of stem cells or their progeny cultured in the presence of an Hsf1 activator or an SCp homeostat for use in the manufacture of a medicament for regenerative medicine, cell therapy, of cellular transplantation.

### EXAMPLES

### Example 1. Hsf1 is Required for HSC Maintenance In Vitro

To test if HSCs depend on Hsf1 for their maintenance in vitro, we conditionally deleted Hsf1 from HSCs, cultured them for 10 days and assessed their long-term multilineage reconstituting activity in competitive transplantation assays. The assay for testing HSC function is to assess their capacity to give long-term (16+ week) multilineage (B cell, T cell and myeloid cell) reconstitution of the blood system of an irradiated transplant recipient. A competitive transplantation assay provides a quantitative readout of HSC regenerative activity.

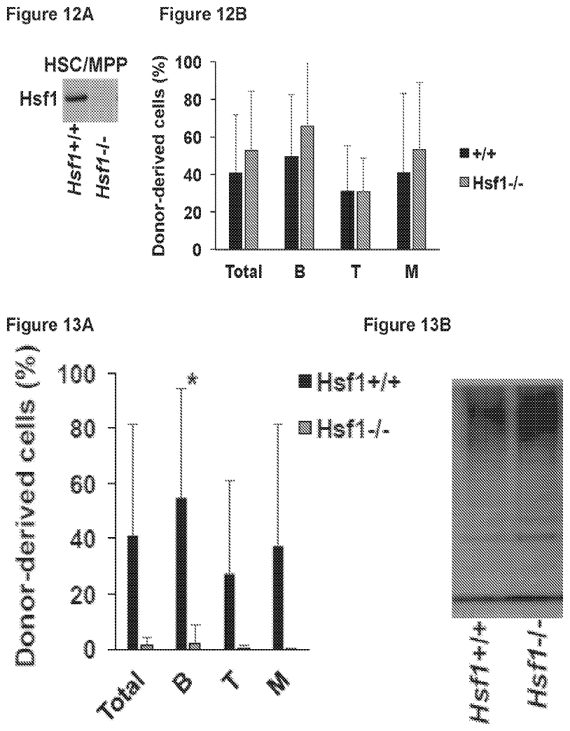

Hsf1^{fl/fl} mice were backcrossed onto a C57BL/6 background, and bred to Mx1-Cre⁺ mice. Mx1 is an interferon responsive promoter induced in hematopoietic cells, including HSCs, after treating mice with polyinosine:polycytidine (pI:pC). We administered pI:pC (10 μg; 3-6 total doses administered every other day) to 2 month old Mx1-Cre⁺;Hsf1^{fl/fl} and Mx1-Cre⁻;Hsf1^{fl/fl} littermate control mice to delete Hsf1. This treatment leads to efficient deletion of Hsf1 within HSCs in vivo (FIG. 12A). Seven days after pI:pC administration, when HSCs returned to steady state, CD150⁺CD48⁻Lineage⁻ckit⁺Sca1⁺ (CD150⁺CD48⁻LSK) HSCs (CD45.2⁺) were sorted (single cell mode) into 96-well plates (10 cells/well) containing 100 uL of cell culture medium.

Culture medium was prepared as follows:

### Reagents:

Prime-XV mouse hematopoietic cell medium (Irvine Scientific)
0.1% BSA (Sigma, Catalog # A2058, Lot #059K1653)
50 ng/mL SCF (Peprotech)
50 ng/mL TPO (Peprotech)
50 uM 2-Mercaptoethanol (Sigma)

### Preparing Components:

BSA: 1 g BSA dissolved in 10 mL water to make a 10% BSA solution. This solution is 100×. Store 1 mL aliquots at −80 C. This can be freeze-thawed.
TPO: Make 50 ug/mL solution in PBS w/0.1% BSA. This solution is 1000×. Store 10 uL aliquots at −80 C.
SCF: Make 50 ug/mL solution in PBS w/0.1% BSA. This solution is 1000×. Store 10 uL aliquots at −80 C.
2-Mercaptoethanol: Dilute 25 uL stock solution in 3.5 mL Media (Dilution A: 100 mM). Dilute 200 uL of Dilution A in 3.8 mL Media (Dilution B 5 mM). This solution is 100×. This should always be made fresh.

### Preparing Media (10 mL):

9.78 mL Prime-XV medium
100 uL BSA (100×)
100 uL 2-Mercaptoethanol (100×)
10 uL TPO (1000×)
10 uL SCF (1000×)

HSCs (CD45.2⁺) were cultured for 10 days at 37° C., 5% CO₂. After 10 days, the entire cellular contents of each well were transplanted with 2×10⁵ freshly isolated congenic bone marrow cells (CD45.1⁺) into irradiated mice (CD45.1⁺). Recipient mice were bled monthly (for 4 months) to assess levels of donor-derived CD45.2⁺ hematopoietic cells. Hsf1-deficient HSCs grown in vitro for 10 days exhibited a significant loss of multilineage reconstituting activity upon transplantation as compared to wild-type controls (FIG. 13A). The blood of transplant recipients of cultured Hsf1-deficient HSCs contained very few cells derived from these Hsf1-deficient HSCs, and rarely contained donor-derived B cells, T cells and myeloid cells. In contrast, recipients of cultured wild-type cells had more donor-derived B cells, T cells, and myeloid cells in their blood. Indeed, Hsf1-deficient cells minimally contributed to the reconstituted blood system of recipient mice.

In contrast to these data generated using HSC cultured in vitro, Hsf1 was not required to promote HSC maintenance or activity in vivo. We competitively transplanted 5×10⁵ freshly isolated Hsf1-deficient or control bone marrow cells together with 5×10⁵ freshly isolated congenic bone marrow cells into irradiated mice. Hsf1-deficient bone marrow gave comparable levels of reconstitution as wild-type controls (FIG. 12B).

Overall, these data indicate that Hsf1 promotes the maintenance of HSCs in vitro.

### Example 2. Hsf1 Promotes the Maintenance of Protein Homeostasis in Cultured HSCs

HSCs accumulate large amounts of ubiquitylated protein in vitro, indicative of a severe imbalance in protein homeostasis (FIG. 10B, 10C). Modest declines in protein homeostasis are sufficient to impair HSC maintenance and function in vivo (FIG. 9). HSC maintenance in vitro depends, at least in part, on Hsf1 (FIG. 13A), the master regulator of the cytosolic proteotoxic stress response. These data indicate that increasing Hsf1 activity could promote HSC maintenance in vitro by maintaining protein homeostasis.

To test if Hsf1 promotes protein homeostasis within HSCs in vitro, we cultured Hsf1-deficient and littermate control HSCs for 18 hours and assessed the abundance of ubiquitylated protein by western blot. In this experiment, we observed increased ubiquitylated protein within Hsf1-deficient HSCs as compared to controls (FIG. 13B). These data demonstrate that Hsf1 promotes protein homeostasis maintenance within cultured HSCs.

### Example 3. Addition of 17-AAG and HSF1A to Culture Medium Promotes Hsf1 Accumulation within Cultured HSCs

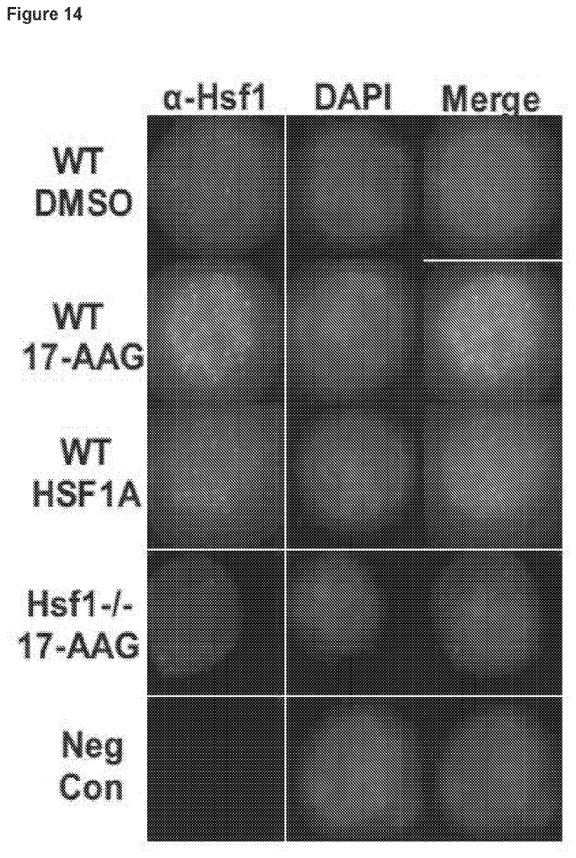

Since Hsf1 promoted HSC maintenance and protein homeostasis in vitro (FIG. 13), we investigated whether increasing Hsf1 expression or activity would further enhance HSC maintenance. Inactive Hsf1 is thought to be primarily localized in the cytoplasm where it binds to chaperones/chaperonins, such as HSP90 and TRiC (FIG. 11). Hsf1 can become active as a transcription factor when it translocates to the nucleus. We thus tested whether inhibitors of HSP90 or TRiC would promote Hsf1 accumulation in the nucleus of cultured HSCs by culturing HSCs for 10 days in the presence or absence of 17-AAG (HSP90 inhibitor; 5 nM final concentration) or HSF1A (TRiC inhibitor; 8 uM final concentration), and assessed Hsf1 expression and localization by immunofluorescence. Addition of either 17-AAG or HSF1A to culture medium was sufficient to increase Hsf1 abundance in the nucleus of cultured HSCs (FIG. 14). These data indicate that HSP90 and TRiC inhibitors induce Hsf1 accumulation in the nuclei of cultured HSCs, where it is active.

### Example 4. Addition of 17-AAG to Culture Medium Reduces Unfolded Proteins in Cultured HSCs

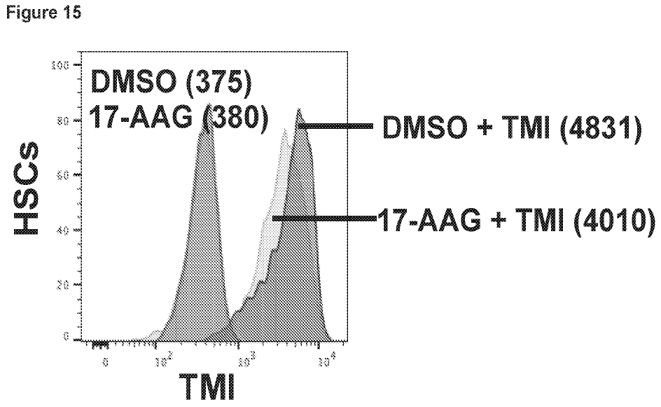

Hsf1 promoted protein homeostasis maintenance in cultured HSCs (FIG. 13B), and 17-AAG promoted Hsf1 accumulation in cultured HSCs (FIG. 14). We thus investigated whether addition of 17-AAG to culture medium could promote protein homeostasis maintenance in HSCs. To test this, HSCs were cultured in the presence or absence (vehicle only; DMSO) of 17-AAG. After 18 hours in culture, we assessed protein homeostasis by quantifying unfolded protein abundance based on TMI fluorescence. HSCs cultured in the presence of 17-AAG exhibited less TMI fluorescence than controls (FIG. 15). These data indicate that HSP90 inhibitors enhance protein homeostasis maintenance within cultured HSCs. A similar result would be expected for TRiC inhibitors (e.g. HSF1A) or other activators of Hsf1.

### Example 5. Addition of 17-AAG or HSF1A to Culture Medium Promotes HSC Maintenance In Vitro

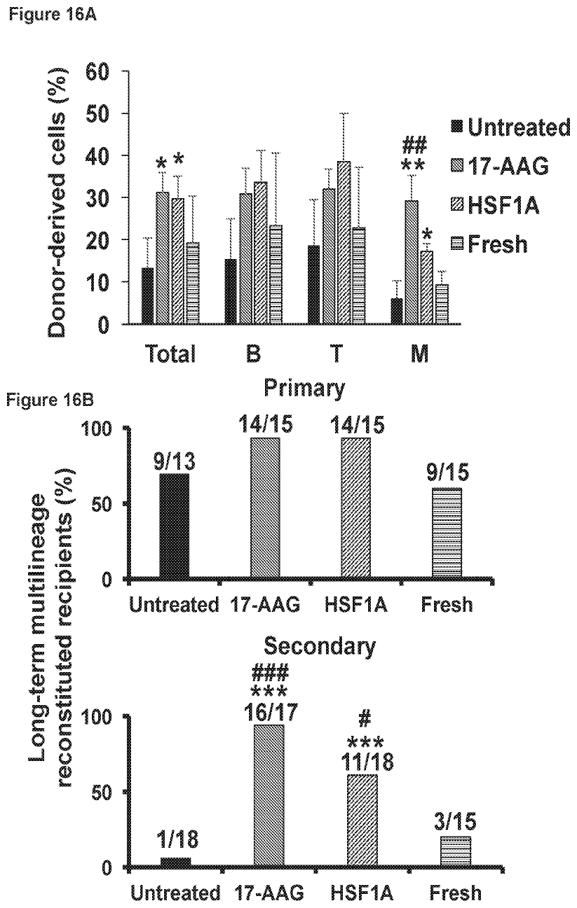

HSCs exhibit severe declines in proteostasis in vitro (FIG. 6). Modest declines in proteostasis impair HSC maintenance in vivo. Our preliminary studies suggest that HSC maintenance in vitro depends, at least in part, on Hsf1 (FIG. 9), the master regulator of the cytosolic proteotoxic stress response. This raises the possibility that increasing Hsf1 activity could promote HSC maintenance in vitro by enhancing proteostasis capacity. To test if HSP90 inhibitors or TRiC inhibitors promote HSC maintenance in vitro, purified HSCs were sorted from young adult murine bone marrow (CD45.1⁺) into 96-well plates (10/well) containing our HSC medium supplemented with 17-AAG or HSF1A. HSCs were cultured for 10 days and competitively transplanted with 2×10⁵ freshly isolated congenic bone marrow cells (CD45.2⁺) into irradiated mice (CD45.2⁺). Recipient mice were bled monthly (for 4 months) to assess levels of donor-derived CD45.1⁺ hematopoietic cells. Self-renewal capacity of HSCs was assessed by serially transplanting hematopoietic cells from the primary recipients into secondary irradiated recipients (3×10⁶ cells/recipient). Primary recipients of HSCs cultured in the presence of 17-AAG or HSF1A exhibited significantly higher levels of donor cell reconstitution as compared to controls (cultured in the absence of 17-AAG or HSF1A) (FIG. 16A), and this effect is even more pronounced in the secondary recipients. In addition, a greater fraction of recipients of HSCs cultured in the presence of 17-AAG or HSF1A exhibited long-term multilineage reconstitution in primary and secondary transplants (FIG. 16B). These data demonstrate that 17-AAG and HSF1A promote HSC maintenance in vitro. Similar results are expected for other HSP90 or TRiC inhibitors.

### Example 6. Addition of 17-AAG or HSF1A to Culture Medium Promotes HSC Expansion In Vitro

To test if HSP90 inhibitors or TRiC inhibitors enable HSC expansion in vitro, purified HSCs were sorted from young adult bone marrow (CD45.1⁺) into 96-well plates (10/well) containing HSC medium supplemented with 17-AAG or HSF1A. HSCs were cultured for 10 days and competitively transplanted with 2×10⁵ freshly isolated congenic bone marrow cells (CD45.2⁺) into irradiated mice (CD45.2⁺). At the same time, 10 freshly isolated HSCs were transplanted with 2×10⁵ freshly isolated congenic bone marrow cells into irradiated mice. Recipient mice were bled monthly (for 4 months) to assess levels of donor-derived CD45.1⁺ hematopoietic cells. Self-renewal capacity of HSCs was assessed by serially transplanting hematopoietic cells (3×10⁶ cells/recipient) into secondary irradiated recipients. Recipients of HSCs cultured in the presence of 17-AAG or HSF1A exhibited higher levels of donor cell reconstitution as compared to recipients of freshly isolated HSCs (FIG. 16A). A significantly greater fraction of recipients of HSCs cultured in the presence of 17-AAG or HSF1A exhibited long-term multilineage reconstitution in secondary transplants as compared to freshly isolated HSCs (FIG. 16B). These data strongly support that 17-AAG and HSF1A enable HSC expansion in vitro. Similar results are expected for other HSP90 or TRiC inhibitors.

### Example 7. The Effects of 17-AAG and HSF1A on HSC Maintenance/Expansion In Vitro Require Hsf1

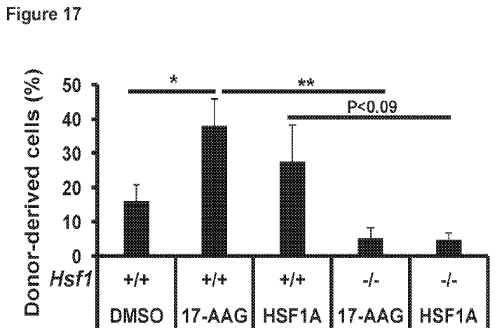

We tested whether the positive effects of 17-AAG and HSF1A on HSC maintenance/expansion in vitro were mediated by Hsf1. Purified Hsf1-deficient and control HSCs were sorted from adult bone marrow (CD45.2⁺) into 96-well plates (10/well) containing HSC medium supplemented with 17-AAG, HSF1A or vehicle (DMSO). HSCs were cultured for 10 days and competitively transplanted with 2×10⁵ freshly isolated congenic bone marrow cells (CD45.1⁺) into irradiated mice (CD45.1⁺). Recipient mice were bled monthly to assess levels of donor-derived CD45.2⁺ hematopoietic cells. Recipients of HSCs cultured in the presence of 17-AAG or HSF1A exhibited higher levels of donor cell reconstitution as compared to controls (FIG. 16A, 17). However, the positive effect of 17-AAG and HSF1A was lost with Hsf1-deficient HSCs (FIG. 17). These data indicate that 17-AAG and HSF1A at least partly promote HSC maintenance/expansion in vitro in and Hsf1 dependent manner. These data support that other agents or mechanisms that induce or activate Hsf1 would be expected to have similar effects on HSC maintenance/expansion.

### Example 8. Addition of 17-AAG to Culture Medium Increases the Frequency and Number of Phenotypic HSCs

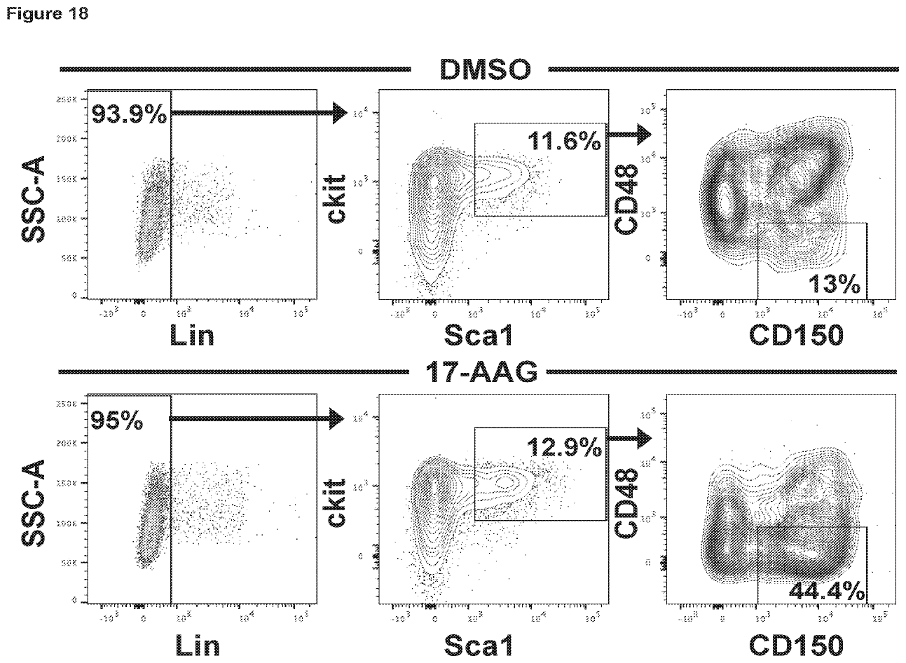

To further test if HSP90 inhibitors promote HSC maintenance/expansion, we cultured purified HSCs (10/well) for 10 days in the presence or absence of 17-AAG, and assessed the frequency of CD150⁺CD48⁻LSK cells in culture. CD150⁺CD48⁻LSK cells were about ˜4-fold enriched when 17-AAG was added to the culture medium (FIG. 18). These data demonstrate that 17-AAG inhibitors promote HSC maintenance and expansion in vitro. Similar results are expected for other HSP90 inhibitors, TRiC inhibitors and agonists of Hsf1.

### Example 9. Addition of 17-AAG to Culture Medium Supports Highly Efficient Genetic Modification of HSCs

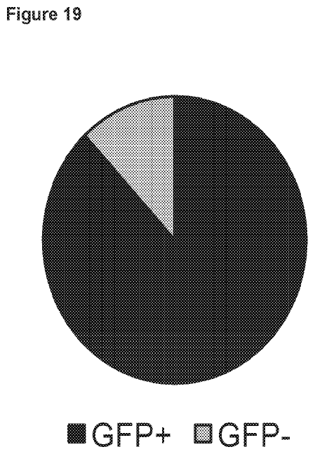

We tested whether the presence of HSP90 inhibitors in cell culture medium permitted efficient genetic modification of HSCs. Purified HSCs were cultured overnight in medium supplemented with 17-AAG. Retroviral supernatant (containing retrovirus encoding MSCV-IRES-GFP) was added two times (two hours apart), and the cells were cultured for an additional 24 hours. After culture, the cells were plated in methylcellulose semi-solid medium supplemented with hematopoietic cytokines. Ten days later, individual colonies (derived from single HSCs) were assessed for GFP expression by flow cytometry. GFP was detected in 22/25 colonies (88%; FIG. 19), indicating that the addition of 17-AAG to culture medium was compatible with highly efficient genetic modification of stem cells. We expect this will also be true for other HSP90 inhibitors, TRiC inhibitors and Hsf1 agonists. In addition, we expect that these agents will also be compatible for genetic modification of stem cells by other technologies (in addition to retrovirus), including but not limited to lentivirus, CRISPR and other genetic modification or editing technologies.

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A method of retaining the regenerative activity of mammalian multipotent stem cells, comprising storing or culturing the multipotent stem cells in the presence of an Hsf1 activator or a stem cell protein (SCp) homeostat, wherein the stem cells retain regenerative activity.

2. The method of claim 1 comprising maintaining mammalian multipotent stem cells in culture.

3. The method of claim 1 comprising expanding mammalian multipotent stem cells in culture.

4. The method of claim 1 comprising storing mammalian multipotent stem cells.

5. A method of preparing mammalian multipotent stem cells or their progeny for transplantation into a mammal, comprising culturing the stem cells in the presence of an Hsf1 activator or a SCp homeostat, wherein the stem cells retain regenerative activity.

6. The method of claim 5 comprising potentiating the transplantation of mammalian cells into a mammal in need of regenerative therapy or cell therapy, by administering multipotent stem cells or their progeny which have been cultured in the presence of an Hsf1 activator or a SCp homeostat, wherein the stem cells or their progeny have improved engraftment in the mammal compared to the same multipotent stem cells and their progeny cultured in the absence of an Hsf1 activator or a SCp homeostat.

7. The method of claim 1 further comprising exposing the stem cells to a genetic modification reagent in vitro, whereby the stem cells are genetically modified.

8. A method of deriving multipotent stem cells from pluripotent stem cells, or progenitor cells or differentiated cells from multipotent stem cells, comprising differentiating the pluripotent stem cells, or multipotent stem cells, in the presence of an Hsf1 activator or SCp homeostat.

9. The method of claim 5, wherein progeny progenitor cells or differentiated cells are generated from multipotent stem cells by differentiating the multipotent stem cells in the presence of an Hsf1 activator or SCp homeostat.

10. The method according to claim 1 wherein the multipotent stem cell is a hematopoietic stem cell, a neural stem cell, a skin stem cell, a muscle stem cell, a germ-line stem cell, a mesenchymal stem cell, a skeletal stem cells, a pancreatic stem cell, a liver stem cell, a cardiac stem cell, a hair follicle stem cell, an endothelial stem cell, an epithelial stem cell, a mammary stem cell, an adipose-derived stem cell, or an intestinal stem cell.

11. The method according to claim 10 wherein the multipotent stem cell is a hematopoietic stem cell.

12. The method according to claim 1, wherein the Hsf1 activator is a SCp homeostat.

13. The method according to claim 1, wherein the Hsf1 activator is an HSP90 inhibitor.

14. The method according to claim 13, wherein the HSP90 inhibitor is 17-AAG.

15. The method according to claim 1, wherein the Hsf1 activator is a TRiC inhibitor.

16. The method according to claim 15, wherein the TRiC inhibitor is HSF1A.

17. The method according to claim 1, wherein the regenerative activity is greater than freshly isolated multipotent stem cells.

18. (canceled)

19. A pharmaceutical composition comprising mammalian multipotent stem cells or their progeny and a pharmaceutically acceptable carrier, wherein the multipotent stem cells have been stored or cultured according to the method of claim 1.

20. (canceled)

21. (canceled)

22. The pharmaceutical composition according to claim 19 wherein the multipotent stem cell is a hematopoietic stem cell, a neural stem cell, a skin stem cell, a muscle stem cell, a germ-line stem cell, a mesenchymal stem cell, a skeletal stem cells, a pancreatic stem cell, a liver stem cell, a cardiac stem cell, a hair follicle stem cell, an endothelial stem cell, an epithelial stem cell, a mammary stem cell or an intestinal stem cell.

23. The pharmaceutical composition according to claim 22 wherein the multipotent stem cell is a hematopoietic stem cell.

24-35. (canceled)
